(19) United States
(12) Patent Application Publication
(10) Pub. No.: US 2011/0182964 A1
(43) Pub. Date: Jul. 28, 2011

(54) Vascular Stent Which Elutes Amino Acid-Methyl-Ester Derivatives for the Treatment of Vulnerable Plaque and Vascular Disease

(75) Inventors: Robert J. Melder, Santa Rosa, CA (US); Stefan Tunev, Santa Rosa, CA (US)

(73) Assignee: Medtronic, Inc., Minneapolis, MN (US)

(21) Appl. No.: 13/012,374

(22) Filed: Jan. 24, 2011

Related U.S. Application Data
(60) Provisional application No. 61/297,371, filed on Jan. 22, 2010.

(51) Int. Cl. A61K 31/195; A61K 9/00; A61K 31/215; A61K 31/216; A61K 31/223; A61P 9/10; A61P 37/06

(52) U.S. Cl. 424/423; 514/561; 514/529; 514/538; 514/551

(57) ABSTRACT

This invention provides implantable medical devices that include delivery systems containing amino acid derivatives, and related methods for making and using. More specifically, this invention provides vascular stents with coatings containing methyl ester derivatives of amino acids, and related methods for making and using.

### RELATED APPLICATIONS

This application claims the benefit under 35 U.S.C. §119(e) of U.S. provisional application Ser. No. 61/297,371, filed Jan. 22, 2010, which is specifically incorporated by reference herein.

### FIELD OF THE INVENTION

This invention relates generally to implantable medical devices that include delivery systems containing amino acid derivatives, and related methods for making and using. More specifically, the present invention relates to vascular stents with coatings containing methyl ester derivatives of amino acids, and related methods for making and using.

### BACKGROUND OF THE INVENTION

Implantable medical devices are used for myriad purposes throughout a subject's body. They can include pace makers, heart valves, vascular stents, catheters, and vascular grafts. Implantable medical devices must be biocompatible to prevent inducing life threatening adverse physiological responses between the implant recipient and device.

Recently, implantable medical devices have gained significant use in, treating circulatory disease caused by a progressive blockage of the blood vessels that perfuse the heart and other major organs. More severe blockage of blood vessels in such individuals often leads to hypertension, ischemic injury, stroke, or myocardial infarction. Atherosclerotic lesions, which limit or obstruct coronary blood flow, are the major cause of ischemic heart disease. Percutaneous transluminal coronary angioplasty is a medical procedure whose purpose is to increase blood flow through an artery. Percutaneous transluminal coronary angioplasty is the predominant treatment for coronary vessel stenosis. The increasing use of this procedure is attributable to its relatively high success rate and its minimal invasiveness compared with coronary bypass surgery. A limitation associated with percutaneous transluminal coronary angioplasty is the abrupt closure of the vessel, which may occur immediately after the procedure, and restenosis, which occurs gradually following the procedure.

Upon pressure expansion of an intracoronary balloon catheter during angioplasty, smooth muscle cells within the vessel wall become injured, initiating a thrombotic and inflammatory response. Cell derived growth factors such as platelet derived growth factor, basic fibroblast growth factor, epidermal growth factor, thrombin, etc., released from platelets, invading macrophages and/or leukocytes, or directly from the smooth muscle cells provoke a proliferative and migratory response in medial smooth muscle cells. Inflammatory cells therefore are present and may contribute to both the acute and chronic phases of restenosis.

Accordingly, the implantation of stents has gained widespread use to maintain increased blood flow; however, in many instances restenosis still occurs, and simply maintaining blood flow does not treat the underlying problem. The vascular response to stent implantation often involves a macrophage-mediated response, giving rise to giant cells which can contribute to a chronic inflammatory response causing neointimal growth and restenosis of the stented vascular segment.

Not only are macrophages involved in restenosis, but they are involved in the process of atherosclerosis, causing the formation of atherosclerotic lesions or plaque. Atherosclerosis is a progressive, dynamic inflammatory disorder characterized by the accumulation of lipids, cells, and extracellular matrix in the vessel walls. One of the early events in atherosclerosis is the entry of monocytes into focal areas of the arterial subendothelium that have accumulated matrix-retained lipoproteins, often including modified lipoproteins. These monocytes differentiate into macrophages and the macrophages accumulate large amounts of intracellular cholesterol through the ingestion of lipoproteins in the subendothelium. Thus, macrophage-mediated inflammation is a major contributor to atherosclerotic disease progression, and macrophage apoptosis is an important feature of atherosclerotic plaque evolution. In early lesions, macrophage apoptosis limits lesion cellularity and suppresses plaque progression while in advanced lesions, macrophages promote the plaque disruption and acute lumenal thrombosis. Further, the cytokine macrophage migration inhibitory factor (MIF) is a unique pro-inflammatory regulator of many acute and chronic inflammatory diseases. MIF is highly expressed by macrophages in atherosclerotic plaques, and contributes to lesion progression and plaque inflammation, reinforcing the inflammatory cascade.

Vulnerable plaque is composed of a thin fibrous cap covering a liquid-like core composed of an atheromatous gruel. The exact composition of mature atherosclerotic plaques varies considerably and the factors that effect an atherosclerotic plaque's make-up are poorly understood. However, the fibrous cap associated with many atherosclerotic plaques is formed from a connective tissue matrix of smooth muscle cells, types I and III collagen and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. The ratio of fibrous cap material to atheromatous gruel determines plaque stability and type. When atherosclerotic plaque is prone to rupture due to instability it is referred to a “vulnerable” plaque. Upon rupture the atheromatous gruel is released into the blood stream and induces a massive thrombogenic response leading to sudden coronary death. Recently, it has been postulated that vulnerable plaque can be stabilized by stenting the plaque.

Drug eluting stents (DES) are known and have been on the market for several years now with excellent clinical success. Drug eluting stents have revolutionized the vascular and cardiologic medicine, aiding in such complications as vulnerable plaque rupture, stenosis, restenosis, ischemic myocardial infarct, and atherosclerosis. However, as with any evolving technology, there is still a need for addressing problems of atherosclorsis.

### SUMMARY OF THE INVENTION

The present invention incorporates amino acid derivatives, particularly amino acid esters, and more particularly amino acid methyl esters, as therapeutic agents designed to induce the death of pro-inflammatory cells, e.g. macrophages, as a means to halt the local involvement of macrophage in the vascular disease process, for example. In particular, they can be used in treating and/or preventing both early and late (potentially vulnerable) atherosclerotic lesions and local response to stenting, for example. They may be eluted from a polymer coating on the stent, or they may also be released directly from pores or compartments within the stent struts. The present invention recognizes that removal of macrophage participation in the treatment of atherosclerotic lesions would limit the progression of local disease, remove the threat of macrophage-mediated plaque rupture, and reduce the local response to implanted stent materials.

The present invention generally provides methods, delivery systems, and implantable medical devices useful for removing macrophages from the atherosclerotic process. Significantly, this can be done using, for example, methyl ester derivatives of amino acids.

The delivery systems of the present invention may include polymeric coating compositions or they may be “polymerless” delivery systems. They may be used as coatings on an implantable medical device. Alternatively, the methyl ester derivatives of amino acids may be housed inside an implantable medical device and delivered, for example, through pores. For example, a macrophage-cidal derivative of an amino acid may be eluted from a polymer coating on a stent, or it may alternatively or additionally be released directly from pores or compartments within the stent struts.

In one embodiment, the present invention provides a medical device comprising an implantable unit and a macrophage-cidal amino acid derivative associated therewith. Typically, the implantable unit is a stent. The stent can be composed of a material selected from the group consisting of metals and polymers. Typically, the implantable unit comprises one or more biodegradable, bioabsorbable, biostable, bioerodable polymers, or combinations thereof.

Typically and preferably, the macrophage-cidal amino acid derivative is an ester of an amino acid, and more preferably, a methyl ester of an amino acid. Examples of such derivatives are the methyl ester derivatives of phenylalanine or leucine, and more typically, phenylalanine.

The present invention also provides methods.

In one embodiment, the present invention provides a method for promoting clearance of macrophages from atherosclerotic lesions, the method comprising contacting an atherosclerotic lesion with a macrophage-cidal amino acid derivative.

In another embodiment, the present invention provides a method for treating atherosclerosis or inhibiting the development of atherosclerosis in a subject, the method comprising contacting a treatment site of the subject with a macrophage-cidal amino acid derivative.

In another embodiment, the present invention provides a method for treating a subject at risk of having or having an atherosclerotic lesion, the method comprising contacting a treatment site of the subject with a macrophage-cidal amino acid derivative.

In another embodiment, the present invention provides a method for reducing foreign body reactions and giant cell responses to an implanted medical device, the method comprising incorporating a macrophage-cidal amino acid derivative in the implanted medical device and implanting the medical device in a subject.

In another embodiment, the present invention provides a method for reducing the foreign body response to an implanted medical device, the method comprising incorporating a macrophage-cidal amino acid derivative in the implanted medical device and implanting the medical device in a subject.

In such methods, the macrophage-cidal amino acid derivative is preferably an ester of an amino acid, and more preferably a methyl ester of an amino acid. The amino acid is preferably phenylalanine or leucine, and more preferably phenylalanine.

In such methods, the macrophage-cidal amino acid derivative is preferably incorporated into a stent. The stent preferably comprises a polymeric coating having the macrophage-cidal amino acid derivative blended therein.

### DEFINITION OF TERMS

Prior to setting forth the invention, it may be helpful to an understanding thereof to set forth definitions of certain terms that will be used hereinafter:

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

As used herein, the term “or” is generally employed in its sense including “and/or” unless the content clearly dictates otherwise. The term “and/or” means one or all of the listed elements or a combination of any two or more of the listed elements (e.g., preventing and/or treating an affliction means preventing, treating, or both treating and preventing further afflictions).

As used herein, all numbers are assumed to be modified by the term “about” and preferably by the term “exactly.” Notwithstanding that the numerical ranges and parameters setting forth the broad scope of the invention are approximations, the numerical values set forth in the specific examples are reported as precisely as possible. All numerical value, however, inherently contain certain errors necessarily resulting from the standard deviation found in their respective testing measurements.

Also herein, the recitations of numerical ranges by endpoints include all numbers subsumed within that range (e.g., 1 to 5 includes 1, 1.5, 2, 2.75, 3, 3.80, 4, 5, etc.).

The term “in the range” includes the endpoints of the stated range.

As used herein, “treatment site” shall mean vascular occlusions, vulnerable plague regions, as well as any site where it is desirable to avoid (or reduce the intensity of) a foreign body response to an implanted device.

As used herein, “animal” or “subject” shall include mammals, fish, reptiles and birds. Mammals include, but are not limited to, primates, including humans, dogs, cats, goats, sheep, rabbits, pigs, horses and cows.

As used herein, “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

As used herein, “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter; an initial gradual release followed by a subsequent burst. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

As used herein, “compatible” refers to a composition possess the optimum, or near optimum combination of physical, chemical, biological and drug release kinetic properties suitable for a controlled release coating made in accordance with the teachings of the present invention. Physical characteristics include durability and elasticity/ductility, chemical characteristics include solubility and/or miscibility, and biological characteristics include biocompatibility. Preferably, the drug release kinetic should be either near zero-order or a combination of first and zero-order kinetics.

As used herein, “drug” or “active agent” or “therapeutic agent” shall include any bioactive agent having a therapeutic effect in an animal. This includes the amino acid derivatives described herein, as well as other active agents that can be used in addition to the amino acid derivatives. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, peroxisome proliferator-activated receptor gamma ligands (PPARγ), hypothemycin, nitric oxide, bisphosphonates, epidermal growth factor inhibitors, antibodies, proteasome inhibitors, antibiotics, anti-sense nucleotides and transforming nucleic acids.

As used herein, a “macrophage-cidal agent” or “macrophage-cidal derivative” refers to an amino acid derivative that induces necrosis or apoptosis of macrophages or macrophage precursors.

As used herein, a macrophage-cidal amino acid derivative is “associated with” with an implantable unit of a medical device if it is somehow incorporated into or onto the implantable unit. For example, it can be incorporated into a polymeric coating on a stent, or it can be within pores or compartments within stent struts, for example, without a polymer.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 depicts a medical device, specifically a vascular stent having the coating made in accordance with the teachings of the present invention thereon.

FIG. 2 depicts a vascular stent having a coating made in accordance with the teachings of the present invention mounted on a suitable delivery device—a balloon catheter.

FIG. 3 depicts a vascular stent 400 having a coating 504 of the present invention mounted on a balloon catheter 601.

### DETAILED DESCRIPTION ILLUSTRATIVE EMBODIMENTS OF THE INVENTION

This invention incorporates amino acid derivatives, particularly amino acid esters, and more particularly amino acid methyl esters, as elutable therapeutic agents designed to induce the death of pro-inflammatory cells, e.g. macrophages, as a means to halt the local involvement of macrophage in the vascular disease process, for example. In particular, they can be used in treating and/or preventing both early and late (potentially vulnerable) atherosclerotic lesions and local response to stenting, for example. They may be eluted from a polymer coating on the stent, or they may also be released directly from pores or compartments within the stent struts.

Macrophage and other inflammatory cells may be destroyed following treatment with methyl ester derivatives of amino acids. In particular, Phenylalanine Methyl Ester (PME), is especially effective in specific lysis of macrophage. Following macrophage exposure to PME, macrophage serine esterases cleave the ester bond resulting in the formation of phenylalanine and methanol, leading to macrophage death but not impacting other normal cell viability or function. PME has been previously employed to induce death of monocyte/macrophage cells in vivo in therapeutic procedures requiring the removal of this cell population.

The present invention involves the site specific delivery of derivatives of amino acids, particularly esters of amino acids, and more particularly methyl esters of amino acids, directly to the treatment site, e.g., a stent deployment area. Site specific delivery is preferred over systemic delivery for several reasons. For example, site specific delivery can achieve high local therapeutic concentration with a small amount of an active agent. Also, site specific delivery can avoid, or reduce the intensity of, unintended side effects at body locations remote from the treatment site. Therefore, administration of the methyl ester derivatives of amino acids described herein directly to the treatment area is preferred.

PME is used commercially as a component in the artificial sweetener, Aspartame, a dipeptide containing PME and aspartic acid. PME is released during the metabolic degradation of Aspartame (N-L-alpha-aspartyl-L-phenylalanine methyl ester). The structures are as follows:

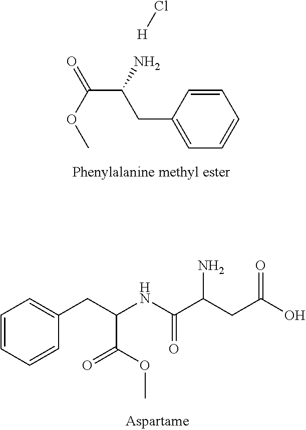

Other methyl ester derivatives of amino acids that can be used according to the present invention include, for example, leucine methyl ester, which as the following structure: (H₃C)₂—CHCH₂CH(NH₂)—C(O)—OCH₃.

Other amino acid derivatives, particularly other esters of amino acids, can also be used as long as they are macrophage-cidal agents. Preferably, such agents are capable of decreasing macrophage activity directly at the site of implantation of the medical device, and more preferably, eliminating substantially all such activity directly at the site.

Such macrophage-cidal agents can be used according to the present invention in methods for promoting clearance of macrophages from atherosclerotic lesions in a subject, treating atherosclerosis or inhibiting the development of atherosclerosis in a subject, treating a subject at risk of having or having an atherosclerotic lesion, reducing foreign body responses and giant cell responses to an implanted stent or other implanted medical device, and reducing restenosis through suppression of foreign body response to an implanted stent or other implanted medical device. The foreign body response is a response of biological tissue to any foreign material in the tissue. Tissue-encapsulation of an implant is part of this. The presence of the implant changes the healing response, which consists of: protein adsorption, macrophages, multinucleated foreign body giant cells (macrophage fusion), fibroblasts, and angiogenesis. A foreign-body giant cell is a collection of fused macrophages (giant cell) which are generated in response to the presence of a large foreign body. This is particularly evident with implants that cause the body chronic inflammation and foreign body response.

Preferably, the amino acid derivatives described herein, and preferably implantable medical devices that incorporate such compounds, are intended to treat physiological and anatomical pathologies in a hemodynamic region of an animal such as the cardiovascular system and the peripheral vascular system. In one preferred embodiment of the present invention, the medical device is a vascular stent used to treat, inhibit, palliate or prevent vascular occlusions and vulnerable plaque.

However, several techniques and corresponding devices, besides stents, are known that can be used to deploy derivatives of amino acids of the present invention, including weeping balloon and injection catheters. Weeping balloon catheters are used to slowly apply an anti-restenotic composition under pressure through fine pores in an inflatable segment at or near the catheter's distal end. The inflatable segment can be the same used to deploy the stent or separate segment. Injection catheters administer the anti-restenotic composition by either emitting a pressurized fluid jet, or by directly piercing the artery wall with one or more needle-like appendage. Recently, needle catheters have been developed to inject drugs into an artery's adventitia. These could be used as well in the present invention.

A wide variety of medical devices, particularly drug eluting stents (DES) are known and can be used to deliver the macrophage-cidal derivatives of amino acids described herein. See, for example, drug delivery stent designs such as those disclosed in U.S. Pat. No. 5,871,535 (Wolff et al.) and U.S. Pat. Pub. No. 2008/0233168 (Cheng et al.). Once positioned at the treatment site the stent or graft is deployed. Generally, stents are deployed using balloon catheters. The balloon expands the stent gently compressing it against the arterial lumen clearing the vascular occlusion or stabilizing the plaque. The catheter is then removed and the stent remains in place permanently.

Many different materials can be used to fabricate the implantable medical devices used to deliver macrophage-cidal amino acid derivatives according to the present invention. These include stainless steel, nitinol, aluminum, chromium, titanium, ceramics, and a wide range of plastics, elastomers, and natural materials including collagen, fibrin and plant fibers. Other specific materials include polyvinylchlorides (PVC), polycarbonates (PC), polyurethanes (PU), polypropylenes (PP), polyethylenes (PE), silicones, polyesters, polymethylmethacrylate (PMMA), hydroxyethylmethacrylate, N-vinyl pyrrolidones, fluorinated polymers such as polytetrafluoroethylene, polyamides, polystyrenes, copolymers or mixtures of these polymers.

In certain embodiments, the present invention provides polymeric delivery systems (e.g., stent coatings and/or filaments) that provide optimized drug-eluting medical device coatings that include macrophage-cidal derivatives of amino acids, particularly methyl ester derivatives of amino acids. The polymers of such systems can be biodegradable, bioabsorbable, biostable, bioerodable, or combinations thereof.

Bioabsorbable polymers include, for example, polylactic acid, polyglycolic acid, polyanhydride, polyphosphate ester, poly-L-lactide, poly-D-lactide, polyglycolide, and combinations thereof, polydioxanone, polycaprolactone, polygluconate, polylactic acid-polyethylene oxide copolymers, modified cellulose, collagen, poly(hydroxybutyrate), polyanhydride, polyphosphoester, poly(amino acids) and combinations or copolymers thereof. Biostable polymers include, for example, silicone, polyurethane, polyester, polypropylene, polyethylene, polytetrafluoroethylene. polycarbonate urethane, and combinations or copolymers thereof.

Preferably, polymers used in accordance with teachings of the present invention provide biocompatible coatings for medical devices intended for use in hemodynamic environments. In one embodiment of the present invention vascular stents are coated using a polymer composition as described herein below. Vascular stents are chosen for exemplary purposes only. Those skilled in the art of material science and medical devices will realize that the polymer compositions described herein are useful in coating a large range of medical devices. Therefore, the use of the vascular stent as an exemplary embodiment is not intended as a limitation.

Furthermore, although the present invention focuses on the delivery of macrophage-cidal derivatives of amino acids (particularly methyl ester derivatives of amino acids) from polymeric coatings of medical devices, those skilled in the art of medical devices and drug delivery will realize that other delivery systems are possible including, for example, systems that release such compounds directly from pores or compartments within a medical device (e.g., from within stent struts).

Vascular stents (hereinafter referred to as “stents”) must be flexible, expandable, and physically stable. Stents are used to relieve the symptoms associated with coronary artery disease caused by occlusion in one or more coronary artery. Occluded coronary arteries result in diminished blood flow to heart muscles causing ischemia induced angina and in severe cases myocardial infarcts and death. Stents are generally deployed using catheters having the stent attached to an inflatable balloon at the catheter's distal end. The catheter is inserted into an artery and guided to the deployment site. In many cases the catheter is inserted into the femoral artery or of the leg or carotid artery and the stent is deployed deep within the coronary vasculature at an occlusion site.

In addition to vascular stents, amino acid derivatives described herein can be associated with (e.g., incorporated into a coating of) implantable medical devices such as vascular stent grafts, urethral stents, bile duct stents, catheters, inflation catheters, injection catheters, guide wires, pace maker leads, ventricular assist devices, and prosthetic heart valves.

Stents and other such medical devices preferably have polymer coatings that are elastic/ductile and adheres to the device surface well. Generally, this requires that coating polymers be amorphous and have glass transition points (Tgs) below body temperature. In addition, polymers used as stent coatings are preferably biocompatible and able to withstand continuous exposure to hemodynamic forces. For example, stent coatings preferably remain non-thrombogenic, non-inflammatory and structurally stable for prolong time periods.

There are many polymer systems that can be used in delivering the amino acid derivatives described herein. Suitable examples are described, for example, in U.S. Pat. Pub. Nos. 2006/0275340 (Udipi et al.) and 2005/0084515 (Udipi et al.). Other examples of polymer systems include phosphorylcholine materials as described in U.S. Pat. No. 5,648,442 (Bowers et al.).

U.S. Pat. Pub. Nos. 2006/0275340 (Udipi et al.) and 2005/0084515 (Udipi et al.) describe miscible polymer blends. Swellabilities of the miscible polymer blends are used as a factor in determining the combinations of polymers for a particular active agent. Also, one of the most fundamental physical chemical properties that must be considered when selecting polymers for use as controlled release coatings is the polymer's solubility parameters and the Tg of the polymers. That is, principles of polymer physical chemistry can be used to match polymeric compositions with drugs so that the resulting controlled release coatings have both optimum physical attributes and drug release kinetic profiles.

Generally, polymers having extremely low Tgs are undesirable when used to coat devices that are subjected to continual hemodynamic forces. As general rule, the lower the Tg the more rubbery a polymer becomes. More rubbery polymers can be tacky and less durable and are more likely to break down when exposed to hemodynamic induced stress and wear than less rubbery ones. This is partially due to the fact that the more rubbery polymers have higher coefficients of friction and possess less structural integrity. Therefore, typically polymers having extremely low Tgs should not be the dominant polymer in polymer blends or copolymer compostions when designing coating polymers intend for stents and other vascular implants. In addition, extremely low Tg (e.g., rubbery) polymers tend to release drugs or bioactive materials at undesirably fast rates due to their high free volumes.

Polymer solubility parameters as a function of a polymer's cohesive properties were known to be a direct expression of the polymer's behavior in aqueous and organic solvents as early as 1916. However, it was not until 1949 that Hildebrand proposed the term solubility parameter and assigned the symbol “8” to represent a polymer's behavior in specific solvents; as previously discussed, “8” will be expressed in J^{1/2}/cm^{3/2}. However, Hildebrand had only considered dispersive forces between various structural units when determining solvent/polymer solubility parameters. Later, Hansen et al. established that the interaction between polar groups and hydrogen bonding contributed significantly to the total cohesive energy, and thus the solubility behavior of many liquids and amorphous polymers. Therefore, Hansen defined a polymer's total solubility (δ_{T}) as the interaction between three distinct values: dispersion force (δ_{D}), polar force (δ_{P}), and hydrogen bonding force (δ_{H}). As used herein δ_{T} will be used to refer to the final solubility parameter of a controlled release coating made in accordance with the teachings of the present invention.

The Hansen solubility parameters can be used to optimize controlled release coating compositions for stents. The optimum drug release kinetic profile occurs when the polymer's solubility parameter closely matches the drug's. However, merely matching candidate drug's solubility with polymer's δ_{T} does not always result in a functional controlled release coating. Various homoploymers, copolymer and combinations thereof, can be designed by balancing the Hansen solubility parameters of the polymer subunits and/or individual polymers in a blend.

A copolymer's biocompatibility, elasticity/ductility and durability can be optimized by altering the ratio of polymeric subunits that favor one property over another. For example, ductility and durability are roughly a function of the polymer's Tg. The lower the Tg, the more ductile the polymer becomes. However, below a certain point the polymer becomes too rubbery and its durability is adversely effected. Moreover, extremely rubbery polymers possess greater first-order kinetics than near zero-order kinetics, consequently, extremely low Tgs are generally avoided.

An exemplary system for controlled release coating design includes a compatible polymer blend designed using these parameters (both δ values and Tg). As used herein compatible polymer blends shall mean two or more chemically distinct polymers, including homopolymers, copolymers and terpolymers that form stable mixtures that do not separate on standing or during prolonged use and possess other desired physical and chemical properties. Methods for compatibilizing two or more polymers with at least one bioactive agent are generally described as follows (and more specifically in U.S. Pat. Pub. Nos. 2006/0275340 (Udipi et al.) and 2005/0084515 (Udipi et al.)).

A first polymer composition known to have certain desirable properties such as biocompatibility and elasticity/ductility is selected. However, the Tg of the first polymer may be below the desired range and thus have poor controlled release properties (for example it may have a first-order kinetic profile). Moreover, the first polymer may not have a δ compatible with the bioactive agent. Consequently, a second polymer composition having solubility parameters and Tg that balance the first polymer's Tg and δ can be blended with the first polymer composition to create an optimum controlled release coating.

Adjustments to the theoretical polymer blends can be made by varying polymer subunit concentrations in accordance with the teachings of the present invention until a δ_{T} approximately equal to the drug's δ is achieved. If the Tg drops below an acceptable range for the drug release kinetics desired the δ_{P} and δ_{D} components can be adjusted, or slightly different polymeric subunits can be selected as necessary. Finally, once the desired Tg range is reached the final concentration of δ_{H} subunits can be adjusted to assure optimum biocompatibility. The final polymer, or polymer blend, will have a δ_{T} approximately equal to the drug's δ and a Tg below body temperature, but not so low as to adversely affect the drug release kinetic profile desired.

In certain embodiments, these parameters are used to create a polymeric coating that has a glass transition point (Tg) of 15° C. to 20° C. In certain embodiments, the polymeric coating is a controlled release coating comprising a polymer component comprising a polymer blend and the macrophage-cidal amino acid derivative wherein the δ_{T} for the macrophage-cidal amino acid derivative is at least 15% greater than the δ_{T} for the polymer component.

In certain embodiments, a compatible polymer blend is made form three polymers, a terpolymer, a copolymer and a homopolymer. In one embodiment the Terpolymer is the low Tg polymer, the copolymer has an intermediate Tg and the homopolymer is the high Tg polymer. In one embodiment the ratio of low Tg polymer to intermediate Tg polymer to high Tg polymer is from approximately 60:20:20 to 80:10:10. In a preferred embodiment the ratio is approximately 67:23:10. The preferred embodiment comprises a terpolymer having a Tg in the range of 15° C. to 25° C., a copolymer having a Tg in the range of 30° C. to 40° C. and a homopolymer having a Tg in the range of 170° C. to 180° C.

Suitable non-limiting exemplary monomers form making such polymers and blends thereof include hydroxy alkyl methacrylate, N-vinyl pyrrolidinone, alkyl methacrylate, vinyl alcohols, acrylic acids, acrylamides, ethylene, vinyl acetate, ethylene glycol di(meth)acrylate, methacrylic acid and terpolymers, co-polymers and homopolymers thereof.

In certain embodiments, the terpolymer preferably comprises relative weight percent concentrations of monomer subunits consisting essentially of vinyl acetate (VAc), an alkyl methacrylate (AMA), and n-vinyl pyrrolidone (NVP). The relative concentrations of each are preferably 1-10% (VAc), 65-80% (AMA) and 10-20% (NVP). The co-polymer comprises monomer subunits consisting essentially of VAc and AMA. The relative concentrations of each are preferably 1-10% (VAc) and 85-99% (AMA). The homopolymer is polyvinyl pyrrolidone. More specifically, the terpolymer comprises the monomer subunits hexylmethacrylate, N-vinylpyrrolidone, and vinyl acetate having a Tg of 15° C. to 20° C., the copolymer comprises the monomer subunits butylmethacrylacte and vinyl acetate having a Tg of 32° C. to 35° C., and the homopolymer (PVP) comprises polyvinylpyrrolidone having a Tg of 174° C.

Release rate is not entirely a function of drug-polymer compatibility. Coating configurations and coating thickness also play roles. When the medical device of the present invention is used in the vasculature, the coating dimensions are generally measured in micrometers (μm). Coatings consistent with the teaching of the present invention may be a thin as 1 μm or a thick as 1000 μm. Generally, if all other physical and chemical factors remain unchanged, the rate at which a given drug diffuses through a given coating is directly proportional to the coating thickness. That is, increasing the coating thickness increases the elution rate and visa versa.

There are at least two distinct coating configurations within the scope of the present invention. In one embodiment of the present invention the drug-containing coating is applied directly to the device surface or onto a polymer primer coating, such a parylene or a parylene derivative. Depending on the solubility rate and profile desired, the drug is either entirely soluble within the polymer matrix, or evenly dispersed throughout. The drug concentration present in the polymer matrix ranges from 0.1% by weight to 30% by weight. In a typical polymeric coating, the macrophage-cidal amino acid derivative is present in the polymer coating at a concentration of 10% to 30% by weight of the polymeric coating. It is most desirable to have as homogenous of a coating composition as possible. This particular configuration is commonly referred to as a drug-polymer matrix.

In another embodiment of the present invention a drug-free polymer barrier, or cap, coat is applied over the drug-containing coating. The drug-containing coating serves as a drug reservoir. Generally, the concentration of drug present in the reservoir ranges from abort 0.1% by weight to as much as 30%, and preferably at a concentration of 10% to 30% by weight. The barrier coating participates in the controlling drug release rates in at least three ways. In one embodiment the barrier coat has a solubility constant different from the underlying drug-containing coating. In this embodiment the drugs diffusivity through the barrier coat is regulated as a function of the barrier coating's solubility factors. The more miscible the drug is in the barrier coat, the quicker it will elute form the device surface and visa versa. This coating configuration is commonly referred to as a reservoir coating.

In another embodiment the barrier coat comprises a porous network where the coating acts as a molecular sieve. The larger the pores relative to the size of the drug, the faster the drug will elute. Moreover, intramolecular interactions will also determine the elution rates. The intramolecular interactions having the greatest net effect on drug elution include the relative hydrophobicity/hydrophilicity (δ_{H}) of the drug-polymer interaction. Generally, the less intramolecular interaction between the drug and polymer barrier coat, the faster the drug will transit the porous network and enter the neighboring tissues. Persons having ordinary skill in the art of material science in combination with the teachings herein will readily understand that many variations on the cap coat and drug-eluting coatings can be made to tune the target diffusivity.

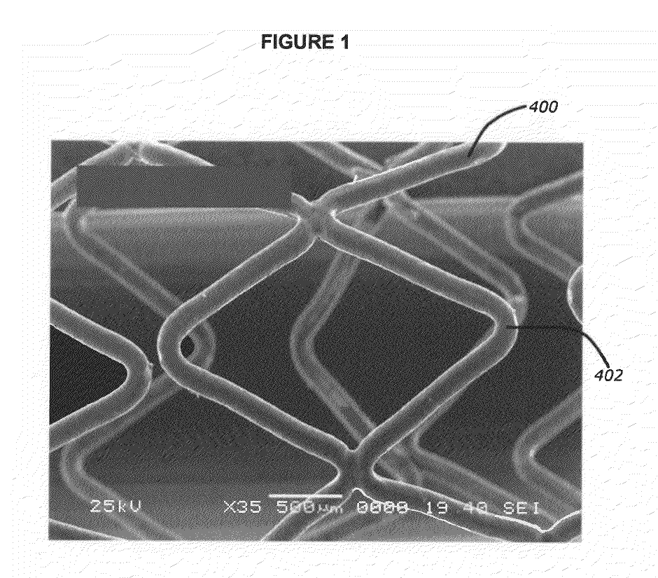

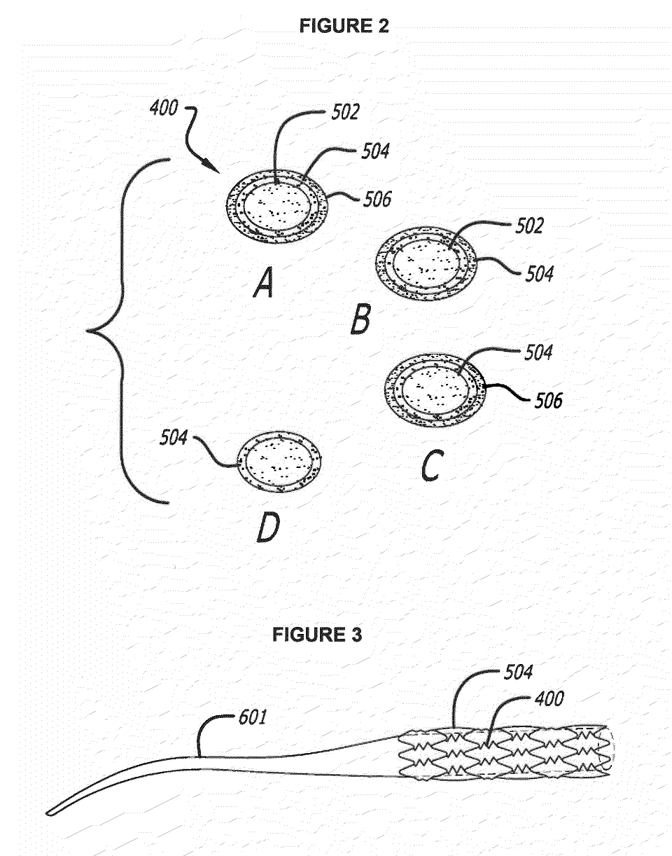

One embodiment of the present invention is depicted in FIG. 1. In FIG. 1 a vascular stent 400 having the structure 402 is made from a material selected from the non-limiting group materials including stainless steel, nitinol, aluminum, chromium, titanium, ceramics, and a wide range of plastics and natural materials including collagen, fibrin and plant fibers. The structure 402 is provided with a coating composition made in accordance with the teachings of the present invention. FIG. 2 depicts a vascular stent 400 having a coating 504 made in accordance with the teachings of the present invention mounted on a balloon catheter 601.

FIG. 2a-d are cross-sections of stent 400 showing various coating configurations. In FIG. 2a stent 400 has a first polymer coating 502 comprising a medical grade primer, such as but not limited to parylene or a parylene derivative; a second controlled release coating 504; and a third barrier, or cap, coat 506. In FIG. 2b stent 400 has a first polymer coating 502 comprising a medical grade primer, such as but not limited to parylene or a parylene derivative, and a second controlled release coating 504. In FIG. 2c stent 400 has a first controlled release coating 504 and a second barrier, or cap, coat 506. In FIG. 2d stent 400 has only a controlled release coating 504.

FIG. 3 depicts a vascular stent 400 having a coating 504 of the present invention mounted on a balloon catheter 601.

The controlled release coatings of the present invention can be applied to medical device surfaces, either primed or bare, in any manner known to those skilled in the art. Applications methods compatible with the present invention include, but are not limited to, spraying, dipping, brushing, vacuum-deposition, and others. Moreover, the controlled release coatings of the present invention may be used with a cap coat. A cap coat as used here refers to the outermost coating layer applied over another coating. For examples, and not intended as a limitation: a metal stent has a parylene primer coat applied to its bare metal surface. Over the primer coat a drug-releasing terpolymer coating or blend of homopolymer, copolymer and terpolymer coating is applied. Over the terpolymer a polymer cap coat is applied. The cap coat may optionally serve as a diffusion barrier to further control the drug release, or provide a separate drug. The cap coat may be merely a biocompatible polymer applied to the surface of the sent to protect the stent and have no effect on elusion rates.

In addition to delivering the amino acid derivatives described herein, other active agents that can be delivered by the systems described herein. Exemplary, non-limiting examples of such other active agents include anti-proliferatives including, but not limited to, macrolide antibiotics including FKBP 12 binding compounds, estrogens, chaperone inhibitors, protease inhibitors, protein-tyrosine kinase inhibitors, peroxisome proliferator-activated receptor gamma ligands (PPARγ), hypothemycin, nitric oxide, bisphosphonates, epidermal growth factor inhibitors, antibodies, proteasome inhibitors, antibiotics, anti-sense nucleotides and transforming nucleic acids.

In one particular non-limiting example the present invention includes a vascular stent having a compatible controlled release coating made in accordance with the present invention. The vascular stent is used to treat an existing vascular occlusion in at least one coronary artery. Stents for stabilizing vulnerable plaque may deploy the macrophage-cidal derivative of an amino acid alone or in combination with anti-inflammatory compounds and/or protease inhibitors, specifically matrix metalloproteinase inhibitors (MMPIs) such as tetracycline-class antibiotics.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A medical device comprising an implantable unit and a macrophage-cidal amino acid derivative associated therewith.

2. The medical device of claim 1 wherein the implantable unit is a stent.

3. The medical device of claim 2 wherein the stent is composed of a material selected from the group consisting of metals and polymers.

4. The medical device of any one of claims 1 through 3 wherein the implantable unit comprises one or more biodegradable, bioabsorbable, biostable, bioerodable polymers, or combinations thereof.

5. The medical device of any one of claims 1 through 4 wherein the implantable unit comprises a polymeric coating having the macrophage-cidal amino acid derivative blended therein.

6. The medical device of claim 5 wherein the macrophage-cidal amino acid derivative is present in the polymer coating at a concentration of 10% to 30% by weight of the polymeric coating.

7. The medical device of any one of claims 1 through 6 wherein the macrophage-cidal amino acid derivative is an ester of an amino acid.

8. The medical device of claim 7 wherein the macrophage-cidal amino acid derivative is a methyl ester of an amino acid.

9. The medical device of claim 8 wherein the amino acid is phenylalanine or leucine.

10. The medical device of claim 9 wherein the amino acid is phenylalanine.

11. A method for promoting clearance of macrophages from atherosclerotic lesions, the method comprising contacting an atherosclerotic lesion with a macrophage-tidal amino acid derivative.

12. A method for treating atherosclerosis or inhibiting the development of atherosclerosis in a subject, the method comprising contacting a treatment site of the subject with a macrophage-cidal amino acid derivative.

13. A method for treating a subject at risk of having or having an atherosclerotic lesion, the method comprising contacting a treatment site of the subject with a macrophage-cidal amino acid derivative.

14. A method for reducing foreign body reactions and giant cell responses to an implanted medical device, the method comprising incorporating a macrophage-cidal amino acid derivative in the implanted medical device and implanting the medical device in a subject.

15. A method for reducing the foreign body response to an implanted medical device, the method comprising incorporating a macrophage-cidal amino acid derivative in the implanted medical device and implanting the medical device in a subject.

16. The method of any one of claims 11 through 15 wherein the macrophage-cidal amino acid derivative is an ester of an amino acid.

17. The method of claim 16 wherein the macrophage-cidal amino acid derivative is an ester of an amino acid.

18. The method of claim 17 wherein the ester of an amino acid is a methyl ester of an amino acid.

19. The method of claim 18 wherein the amino acid is phenylalanine or leucine.

20. The method of claim 19 wherein the amino acid is phenylalanine.

21. The method of any one of claims 11 through 20 wherein the macrophage-cidal amino acid derivative is incorporated into a stent.

22. The method of claim 21 wherein the stent comprises a polymeric coating having the macrophage-cidal amino acid derivative blended therein.
